(19) United States
(12) Patent Application Publication
(10) Pub. No.: US 2013/0173007 A1
(43) Pub. Date: Jul. 4, 2013

(54) SHOULDER PROSTHESIS CUP INCLUDING SECURING RIBS

(71) Applicant: Marc Duport, Toulouse (FR)

(72) Inventor: Marc Duport, Toulouse (FR)

(21) Appl. No.: 13/823,371

(22) PCT Filed: Sep. 15, 2011
(86) PCT No.: PCT/FR11/52117

§ 371 Date: Mar. 14, 2013

(30) Foreign Application Priority Data
Sep. 17, 2010 (FR) 10/57435

(51) Int. Cl. A61F 2/40

(52) U.S. Cl. CPC A61F 2/4081
USPC 623/19.11

(57) ABSTRACT

The invention relates to a prosthesis cup (1), in particular for a shoulder prosthesis, comprising a shell (2) substantially in the shape of a hollow spherical cap defining a concave inner surface (3). The shell (2) includes anchoring means which project from the inner surface (3) and are shaped to penetrate a bone and anchor the cup (1) therein. The invention is characterised in that the anchoring means are shaped to trigger the rotation of the cup (1) as they penetrate the bone.

### TECHNICAL FIELD

The present invention relates to a prosthesis cup, in particular a shoulder prosthesis cup.

### RELATED ART

In France, approximately 15,000 shoulder prostheses are placed each year, including total shoulder prostheses, called standard prostheses, reverse prostheses, and more recently resurfacing prostheses.

This figure, which shows a clear increase in recent years, is in particular due to the increase in the number of pathologies for which the placement of a shoulder prosthesis is now indicated.

In this context, prosthetic shoulder surgery remains a very specialized, highly technical surgery, requiring a long learning period and the use of ancillaries that are often complex. It is therefore desirable to simplify the prostheses and ancillaries so as to facilitate the procedure, make it more reproducible, and therefore decrease postoperative complications. Such a simplification of the ancillary and the procedure may also have the advantage of decreasing the costs of those operations to place shoulder prostheses.

Several types of shoulder prosthesis exist. Certain traditional prostheses, like that described in US 2004/153161, are fixed on the humerus, using a humeral rod, after a resulting resection of the bone. The installation of a humeral rod requires a significant recess in the bone to receive it, thereby decreasing the bone capital and making the bone more fragile. The placement of such a prosthesis is long and complex. After the rod is installed in the recess, the space between the bone and the humeral rod is generally filled with cement. With time, the cement may become dislocated and no longer ensure correct fixing of the rod.

Resurfacing prostheses also exist, like that described in FR 2,928,827, which are placed by epiphysis of the humerus after potential light planing of the surface of the epiphysis. Such prostheses are not necessarily anchored in the bone as previously described.

When a simple resurfacing of the epiphysis is not indicated, for example if the bone is too fragile, the surgeon may choose to use an intermediate so-called “half-resurfacing” technique where part of the epiphysis is resected. The surgeon is then called upon to use a prosthesis like that described in document EP 0,538,895, including a cup designed to replace the part of the epiphysis that is resected and an anchoring screw ensuring fastening of the cup on the bone.

It is also known from document WO 2008/146124 to replace the anchoring screw previously described with a substantially cylindrical anchoring pin. The cup is then provided with a coupling stem cooperating by interlocking with the cavity, with a complementary cylindrical shape, formed to that end in the anchoring pin. Such a cup is then kept in place owing to the pressure exerted by the glenoid. However, the nature of the coupling of the cup and the anchoring pin does not make it possible to avoid rotation of the cup after it is implanted, which can cause problems. Furthermore, the cup simply being interlocked in the cavity of the anchoring pin, the anchoring of the cup on the humeral head may prove insufficient, and thereby cause pain or discomfort for the patient.

All of the known cups are specifically suitable for a specific surgical technique. The surgeon must therefore learn the different techniques and must have several ancillaries adapted to each technique. Furthermore, if the surgeon encounters difficulties during an operation, it is not possible for him to adapt the prosthesis, for example by changing the implantation method of the cup.

### BRIEF SUMMARY

The present invention aims to resolve all or some of the various aforementioned drawbacks.

In this context, the present invention aims to propose an adaptable cup making it possible to use different techniques (traditional, resurfacing, and semi-resurfacing), which preserves the bone capital of the patient and guarantees correct anchoring of the cup, while preventing rotation thereof after the operation.

To that end, the invention relates to a prosthesis cup, in particular for a shoulder prosthesis, comprising a shell substantially in the shape of a hollow spherical cap defining a concave inner surface, the shell including anchoring means that project from the inner surface and are shaped to penetrate a bone and anchor the cup therein, characterized in that the anchoring means are shaped to trigger the rotation of the cup as they penetrate the bone.

Thus, a cup according to the invention makes it possible to guarantee simple and quick anchoring when the resurfacing or half-resurfacing technique is used. The anchoring means, by penetrating the bone, are shaped to impart a rotation to the cup: this rotation results in guaranteeing uniform anchoring of the cup, guaranteeing better resistance to pulling out of the cup, and preventing the cup from rotating once it is implanted. The anchoring means provide better resistance to pulling out of the cup according to the invention.

According to one embodiment, the anchoring means include at least one rib. Each rib increases the anchoring of the cup in the bone and/or improves the resistance to pulling out of the cup. Each rib also makes it possible to prevent the cup from rotating once it is placed on the patient. Advantageously, each rib is integral with the shell and is therefore easy to make.

According to one possibility, each rib has at least one sharp edge with a substantially helical portion. Such a substantially helical portion is easy to produce and may behave like a threading in the bone.

According to one feature, the anchoring means are shaped to cause the cup to rotate by approximately 10° upon impaction.

According to one possibility, each rib has one end situated near the edge of the shell.

According to one embodiment, the cup also includes a coupling stem extending from the inner surface, designed to allow coupling of the cup with a fastening element, in particular an anchoring screw or an anchoring pin.

According to one possibility, the cup also includes a wall, extending from the inner surface and substantially in the shape of a ring arranged concentrically around the coupling stem. Thus, a cup according to the invention is adaptable: The coupling stem makes it possible, depending on the surgeon's needs, to couple the cup to a fastening element. The wall makes it possible on the one hand to anchor the cup directly in the bone to improve the fastening thereof, and on the other hand, depending on the needs, to couple the cup with an intermediate member having a ring portion shape and suitable for being received in the recess delimited between said wall and the coupling stem. Such an intermediate member may, for example, be a coupling member for coupling the cup with an anchoring screw or with an intermediate band making it possible to space the cup away from the bone for better adaptation of the prosthesis on the patient. The wall therefore allows the surgeon to choose the technique he will use to fasten the cup on the patient and possibly to change techniques during the operation. Given the possible compatibility between the anchoring pin and the intermediate member, the surgeon also has the possibility of using both at the same time.

According to one feature of the invention, the wall is crenulated. A crenulated wall makes it possible to improve the anchoring of the cup in the bone. The crenulation also makes it possible to prevent the cup from rotating after it is inserted in the bone. Lastly, the crenulated wall prevents any intermediate member that may have been used from rotating.

According to one embodiment, the wall has a thickness that decreases as it moves away from the inner surface. The decrease in the thickness of the wall moving away from the inner surface gives the wall an improved capacity to penetrate the bone.

According to one embodiment, the wall delimits, with the coupling stem, a recess configured to receive an intermediate member having a substantially tapered outer surface.

According to one possibility, the shell also has at least one notch arranged on the edge of the shell. Such a notch is useful for fastening a tool designed to keep the cup in position and to cause a 10° rotation upon impaction.

### BRIEF DESCRIPTION OF THE DRAWINGS

The invention will be well understood using the following detailed description thereof provided in light of the appended drawing, showing, as one non-limiting example, one embodiment of a cup, in which:

FIG. 1 is a diagrammatic perspective view of a cup;

FIG. 2 is a diagrammatic perspective view of the cup of FIG. 1 coupled with an anchoring pin;

FIG. 3 is a diagrammatic perspective view of the cup of FIG. 1 coupled with an intermediate band and an anchoring pin;

FIG. 4 is a diagrammatic perspective view of the cup of FIG. 1, a coupling member and an anchoring screw for fastening the prosthesis.

### DETAILED DESCRIPTION

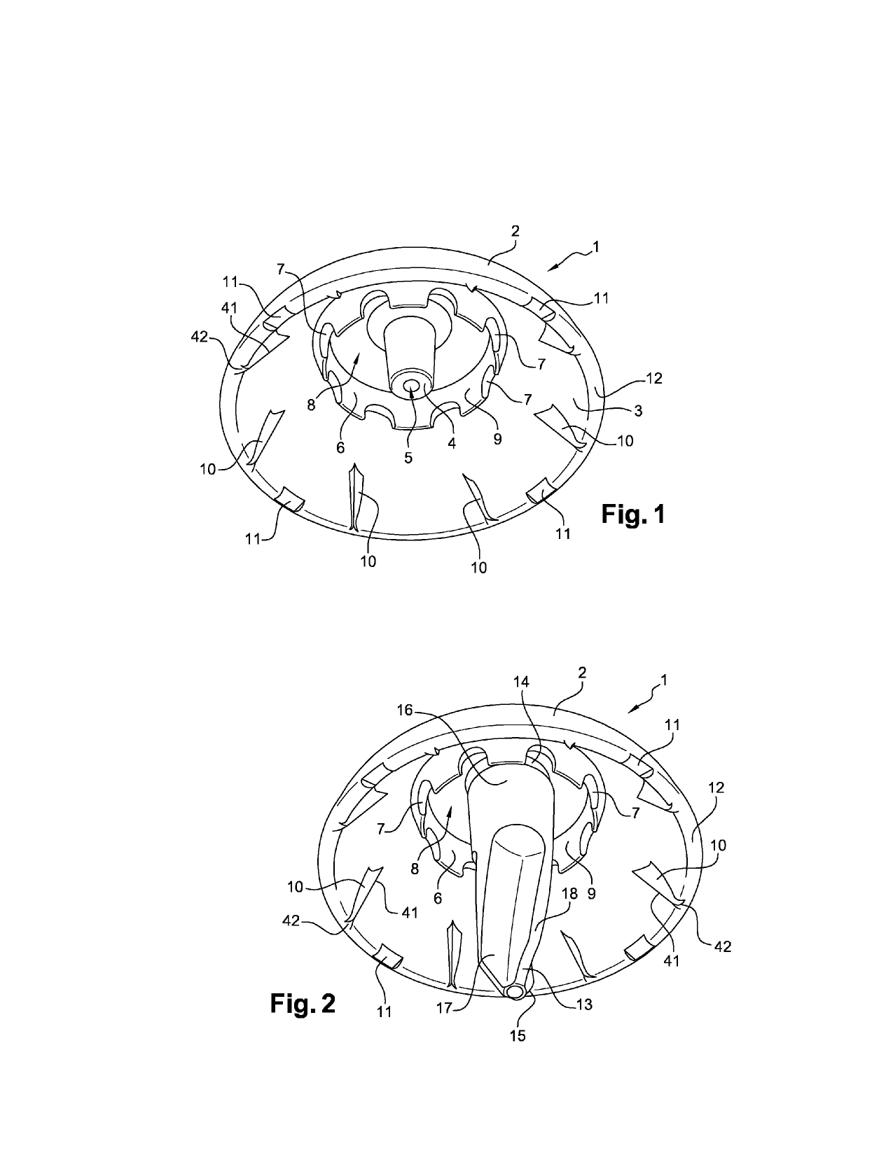

A prosthesis cup 1, illustrated in FIG. 1, includes a shell 2 substantially in the shape of a hollow spherical cap. The shell 2 delimits a concave inner surface 3 and has a coupling stem 4 that extends from that inner surface 3 at the pole of the shell 2. The coupling stem 4 is, for example, provided with a through orifice 5 extending along the axis of the spherical cap. The cup 1 also includes a wall 6 that extends from the inner surface 3 and has a substantially tubular shape. This wall 6 includes eight gaps 7 formed at the free end thereof. The number of gaps 7 may of course be adapted as needed or depending on the size of the cup 1. Likewise, the shape of each gap 7 is adaptable and can, for example, be rounded, as illustrated in FIG. 1, or rectangular. Of course, the depth of each gap 7 may vary until it is substantially equal to the height of the wall 6. The thickness of the wall 6 decreases as it moves away from the inner surface 3. The wall 6 is arranged concentrically with the coupling stem 4, such that they delimit a recess 8 substantially in the shape of a ring. The face 9 of the wall 6 arranged across from the coupling stem 4 has a female tapered shape.

The cup 1 illustrated in FIG. 1 also has anchoring means protruding from the inner surface 3 configured to penetrate the bone while causing the cup 1 to rotate upon the penetration of said anchoring means. Such anchoring means for example include eight ribs 10 formed on the inner surface 3 and integral with the cup 1. The number of ribs 10 can of course be adapted depending on the needs or depending on the size of the cup 1. Each rib 10 extends along a unique longitudinal axis, off-centered relative to the axis of the spherical cap. Each rib 10 has a sharp edge 41, arranged at the free end of the rib 10, having a substantially helical portion 42. The substantially helical portion 42 of the sharp edge 41 is chosen so that the penetration thereof in the bone, during anchoring of the cup 1, facilitates a 10° rotation of the cup 1 during impaction. Lastly, each rib 10 is arranged so as to have one end situated near the edge 12 of the shell 2. Advantageously, each of the ends situated near the edge 12 of the cup 1 is tapered so as to form a harpoon in order to increase the resistance to pulling out of the cup 1.

Lastly, the cup 1, illustrated in FIG. 1, for example includes four notches 11 arranged on the edge 12 of the shell 2.

A surgeon can implant the cup 1 in several ways outlined below, depending on his needs.

The surgeon first has the option of using an anchoring pin 13, illustrated in FIG. 2, to implant the cup 1, for example in the context of resurfacing of the epiphysis of the bone.

The anchoring pin 13 extends along a longitudinal axis between a first end 14, designed to cooperate with the cup 1, and a second end 15, designed to cooperate with the bone. It has a generally tapered shape. The anchoring pin 13 includes a first portion 16 designed to allow coupling of the cup 1. This first portion 16 has a coupling orifice, not shown, extending along the longitudinal axis of the pin 13 and emerging on the first end 14. The shape of this coupling orifice complements the shape of the coupling stem 4 of the cup 1, so as to allow interlocking thereof in the coupling orifice of the pin 13, to couple the cup 1 and the pin 13. Advantageously, the coupling stem 4 and the coupling orifice are tapered. For example, the coupling stem 4 forms a male Morse cone, and the coupling orifice of the pin 13 is a complementary female Morse cone.

The pin 13 also includes a second portion 17 designed to anchor the pin in a bone. To that end, this second portion 17 includes three helical sharp edges 18 allowing the pin 13 to penetrate the bone like a drill while causing the pin 13 to rotate. This rotation of the pin 13 favors its anchoring and increases the resistance of said pin 13 to pulling out. Advantageously, the pin 13 is shaped to cause the cup 1 to rotate in the same direction of rotation as each rib 10.

When the anchoring pin 13 is used to fix the cup 1, the surgeon first fixes the pin 13 in the epiphysis of the bone, then couples the cup 1 thereon by inserting the coupling stem 4 into the coupling orifice of the pin 13. The wall 6 and the ribs 10 are then pushed into the epiphysis of the bone to ensure additional anchoring of the cup 1.

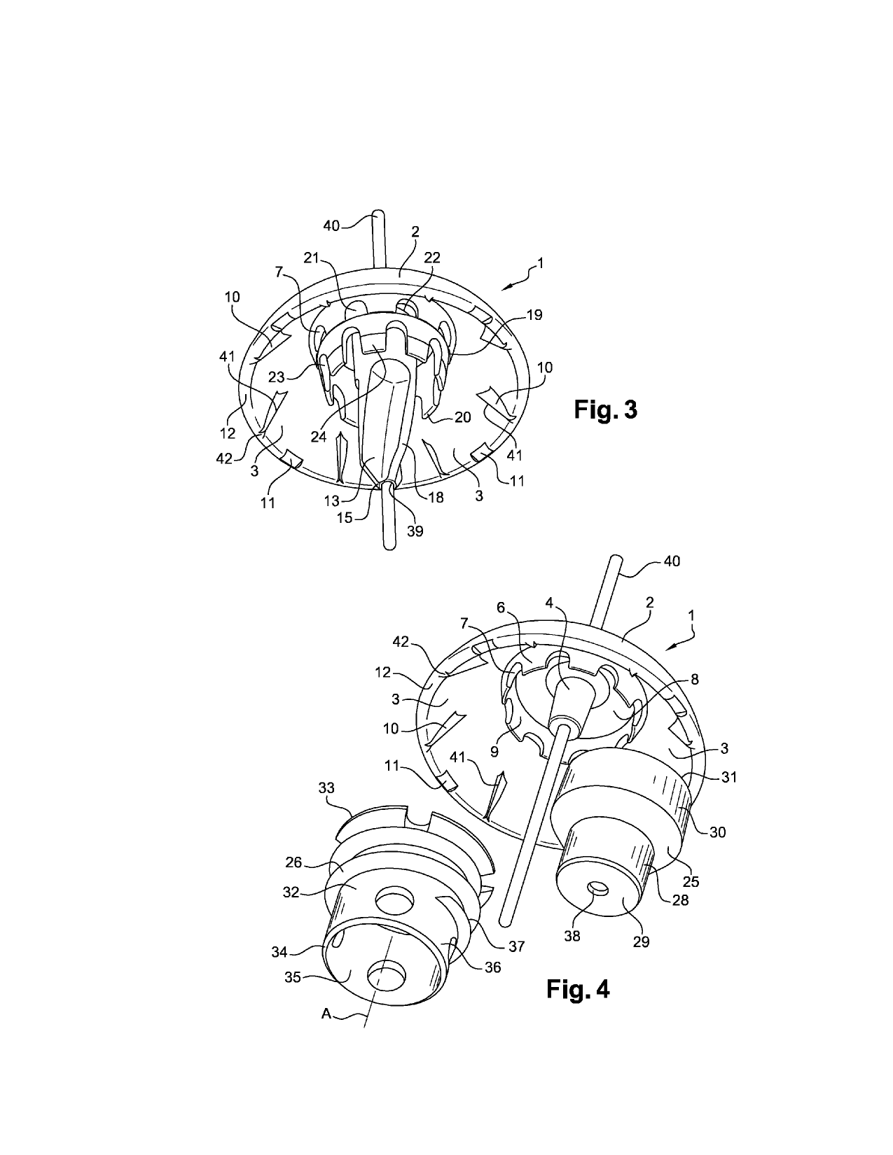

The wall 6 has a height not exceeding 10% of the radius of the spherical cap. For example, the height of the wall 6 is smaller than 7 mm for a radius of the spherical cap of approximately 100 mm. When the epiphysis of the bone has been partially or completely resected, the height of the wall 6 may prove insufficient to be anchored in the bone. The surgeon then has the option of using an intermediate band 19 illustrated in FIG. 3. Such an intermediate band 19 has a tubular body 20 extending along a longitudinal axis between two opposite ends. The tubular body 20 comprises a coupling portion 21 having a shape suitable for being inserted into the recess 8 of the cup 1, such that the outer surface 22 of that coupling portion 21 is in contact with the face 9 of the wall 6 arranged across from the coupling stem 4, to ensure coupling without play of the cup 1 and the intermediate ring 19. The coupling portion 21 may optionally include a stop, not shown, protruding from the outer surface 22, said stop being designed to cooperate with one of the gaps 7 of the cup 1 to lock the rotation of the intermediate band 19 in the recess 8.

The intermediate band 19 also has an anchoring portion 23 including an extension wall 24 having a shape similar to the wall 6. Thus, the extension wall 24 constitutes an extension of the wall 6 and is designed to be anchored in the bone in the same way as the wall 6. Thus, the intermediate band 19 can be anchored in the bone, the cup 1 being coupled thereon. When the intermediate band 19 is coupled with the cup 1, the free end of the intermediate band 19 is situated at a distance from the pole of the spherical cap of approximately 100% of the radius thereof. Thus, the free end of the intermediate band 19 is flush with the edge 12 of the shell 2, such that the intermediate band 19 has a maximum height to favor anchoring of the cup 11 making it possible to completely remove the cup 1 by sawing the bone using a saw guided by the edge 12 of the shell 2.

The surgeon can also use a coupling member 25 and an anchoring screw 26 to fix the cup, illustrated in FIG. 4.

The coupling member 25 includes a first portion 27 with a substantially tapered shape having a male intermediate mounting portion 28 forming a male Morse cone 29. The coupling member 25 also has a second tubular portion 30 provided with a cavity, not shown, that is substantially tapered and delimits a female intermediate mounting portion. The female intermediate mounting portion forms a female Morse cone, not shown, sized to cooperate by shape matching with the coupling stem 4 of the cup 1. According to the embodiment shown in figures, the coupling member 25 has a male Morse cone 29 and a female Morse cone whereof the respective axes of symmetry are combined. Alternatively and not shown, the male Morse cone 29 and the female Morse cone can have distinct respective axes of symmetry that are offset and/or not parallel. The outer surface 31 of the second tubular portion 30 has a shape suitable for being inserted into the recess 8 of the cup 1, such that the outer surface 31 of that second tubular portion 30 is in contact with the face 9 of the wall 6 arranged across from the coupling stem 4, to ensure coupling without play of the cup 1 and the coupling member 25.

The anchoring screw 26, illustrated in FIG. 4, includes a tubular body 32 extending along a longitudinal axis A between a proximal end 33, designed to cooperate directly or indirectly with the cup 1 or the coupling member 25, and an opposite distal end 34 designed to be fixed in the epiphysis of the bone. The tubular body 32 delimits an inner peripheral surface 35, illustrated in FIG. 4, and an outer peripheral surface 36 provided with screwing means, made in the form of a threading 37, to allow the screw 26 to be screwed into the bone.

The inner peripheral surface 35 delimits a female mounting portion, not shown, that extends substantially from the proximal end 33 and that is sized to cooperate by shape matching with the male Morse cone 29 of the coupling member 25. In one embodiment not shown in the figures, the female mounting portion is sized to cooperate by shape matching with the coupling stem 4 of the cup 1 and to be inserted directly into the recess 8.

Thus, the anchoring screw 26 described above makes it possible to fix the cup 1. When the tubular body 32 of the screw 26 is fixed in the bone, the correct anchoring of the screw 26 is ensured using the threading 37 and the placement procedure is simplified, since the coupling of the cup 1 on the screw 26 directly or indirectly with the coupling member 25 is done by simple interlocking.

In order to facilitate the positioning of the cup 1 and the anchoring pin 13 or the coupling member 25, the latter each include a through orifice 38, 39 extending along the longitudinal axis of the coupling member 25 or the anchoring end 13, respectively. Thus, irrespective of the method used to implant the cup 1, the surgeon has the option of using a rod 40, illustrated in FIGS. 3 and 4, to align the cup 1 and the anchoring pin 13 or the coupling member 25. The rod 40 is simply inserted into the through orifice 5 of the cup 1 and into the through orifice 39 of the anchoring pin 13 or into the through orifice 38 of the coupling member 25. The rod 40 may then be inserted into an orifice formed to that end in the bone to guarantee correct orientation of the cup 1.

Thus, the cup 1 according to the invention described above is extremely adaptable to the various surgical techniques commonly used to place a shoulder prosthesis. The wall 6 and the ribs 10 make it possible to anchor the cup 1 directly in the bone. The wall 6 also makes it possible, depending on the surgeon's needs, to couple the cup 1 to a coupling member 25 or an anchoring screw 26 assuming the shape of a ring portion adapted to be received in the recess 8 delimited between the wall 6 and the coupling stem 4. The ribs 10, by penetrating the bone, are shaped to impart a rotation of the cup 1: this rotation results in guaranteeing uniform anchoring of the cup 1, ensuring better resistance of the cup 1 to pulling out, and preventing the cup 1 from rotating once it is implanted.

Of course, the example embodiment described above is in no way limiting, and other details and improvements may be made to the cup 1 according to the invention, without going beyond the scope of the invention, where other forms of cup may be considered.

1. A prosthesis cup, in particular for a shoulder prosthesis, comprising a shell substantially in the shape of a hollow spherical cap defining a concave inner surface, the shell including anchoring means that project from the inner surface and are shaped to penetrate a bone and anchor the cup therein, wherein the anchoring means are shaped to trigger the rotation of the cup as they penetrate the bone.

2. The cup according to claim 1, wherein the anchoring means include at least one rib.

3. The cup according to claim 2, wherein each rib has at least one sharp edge with a substantially helical portion.

4. The cup according to claim 1, wherein the anchoring means are shaped to cause the cup to rotate by approximately 10° upon impaction.

5. The cup according to claim 2, wherein each rib has one end situated near the edge of the shell.

6. The cup according to claim 1, wherein the cup also includes a coupling stem extending from the inner surface, designed to allow coupling of the cup with a fastening element, in particular an anchoring screw or an anchoring pin.

7. The cup according to claim 6, wherein the cup also includes a wall, extending from the inner surface and substantially in the shape of a ring arranged concentrically around the coupling stem.

8. The cup according to claim 7, wherein the wall is crenulated.
